(19) United States
(12) Patent Application Publication
(10) Pub. No.: US 2026/0097241 A1
(43) Pub. Date: Apr. 9, 2026

(54) ULTRASOUND PROBE INCLUDING ULTRASOUND TRANSDUCER WHICH IS MOVABLE ALONG CURVED LINE AND DRIVING METHOD THEREOF

(71) Applicant: NEWPONG CO., LTD, Wonju-si (KR)

(72) Inventors: Jae-Yong LEE, Seoul (KR); Young-Hwan OH, Seoul (KR); Kwang-Jae LEE, Seoul (KR)

(21) Appl. No.: 19/382,268

(22) Filed: Nov. 7, 2025

Related U.S. Application Data
(63) Continuation of application No. PCT/KR2024/012319, filed on Aug. 20, 2024.

(30) Foreign Application Priority Data
May 28, 2024 (KR) 10-2024-0069479

(51) Int. Cl. A61N 7/02

(52) U.S. Cl. CPC A61N 7/02; A61N 2007/025

(57) ABSTRACT

Provided is to an ultrasound probe including an ultrasound transducer which moves in a curved line. The ultrasound probe including an ultrasound transducer which moves in a curved line may include a housing including a contact surface which is provided in a curved shape and is in contact with a target area of a user; a transducer which is provided in the housing and outputs an ultrasound signal; and a movement module which moves the transducer in a curved line in accordance with the curved shape.

### TECHNICAL FIELD

The present disclosure relates to an ultrasound probe including an ultrasound transducer which moves in a curved line.

### BACKGROUND ART

Ultrasound generally refers to a sound wave having a frequency exceeding 20 kHz, which is beyond an audible frequency range perceivable by the human ear, and such ultrasound is widely utilized in ultrasound imaging devices for obtaining images of the inside of an object. Such an ultrasound imaging device is small in size, inexpensive, capable of real-time display, and free from radiation exposure such as X-rays, thereby providing high safety, and is therefore widely used together with other imaging diagnostic devices, such as a computed tomography (CT), a magnetic resonance image (MRI), and a nuclear medicine device.

Along with the ultrasound imaging devices, the technical development of an ultrasound therapy system is also actively being conducted in recent years. Most ultrasound therapy systems are configured to irradiate the body with ultrasound having a frequency of several MHz for medical use, thereby inducing vibration or heat in internal tissues of the body to achieve therapeutic effects. A representative example of such an ultrasound therapy system is a high intensity focused ultrasound system. Generally, the high intensity focused ultrasound system includes a transducer that emits ultrasound, and generates heat by focusing the emitted ultrasound onto a focal point to induce a rapid temperature rise at a portion to be treated. Through such a thermal function, the intended medical procedure can be performed on various affected areas without causing side effects.

Meanwhile, in accordance with the recent increase in interest in beauty, various medical and technical approaches have been attempted for the breakdown of subcutaneous fat. With regard to this, technical approaches for breaking down subcutaneous fat layers through therapeutic ultrasound have been attempted, but further technological development is required to achieve improved therapeutic effects.

In addition, a high intensity focused ultrasound generating device has been proven to be effective in improving skin wrinkles, and is attracting attention as an alternative to invasive procedures such as a face-lifting surgery. The structure of human skin is configured by an epidermal layer, a dermal layer, a subcutaneous fat layer, a muscle layer, and a skeleton in this order from the outside. Among these, most of the components constituting the dermal layer are collagen, which plays a role in maintaining skin elasticity.

The high intensity focused ultrasound does not act on the epidermal layer, but acts on the superficial musculo-aponeurotic system (SMAS) layer which is a part of the muscle layer to not only induce coagulation, but also deliver heat via ultrasound to a deeper region of the dermal layer. As a result, collagen regeneration is promoted, thereby achieving not only wrinkle reduction but also improvement of skin elasticity.

When the position of the transducer moves, the position of the ultrasound signal relative to the skin changes. However, as the ultrasound probe contacts the skin, the compression of the skin according to the shape of the contact area may cause the focal depth within the skin tissue layers to vary depending on the focal position.

A related art of the present disclosure is disclosed in Korean Registered Patent Publication No. 10-1750444.

### DISCLOSURE

### Technical Problem

The present disclosure is made in an effort to solve the problem of the related art as described above and an object is to provide an ultrasound probe including a transducer which has one surface contact with a target area of user which is formed in a curved shape and moves in a curved line in accordance with the curved shape to allow an ultrasound signal to reach to a tissue layer at the same depth.

However, objects to be achieved by the embodiments of the present disclosure are not limited to the technical objects as described above and other technical objects may be present.

### Technical Solution

As a technical means to achieve the above-described technical object, according to an aspect of the present disclosure, an ultrasound probe including an ultrasound transducer which moves in a curved line may include a housing including a contact surface which is provided in a curved shape and is in contact with a target area of a user; a transducer which is provided in the housing and outputs an ultrasound signal; and a movement module which moves the transducer in a curved line in accordance with the curved shape.

According to an exemplary embodiment of the present disclosure, the movement module may further include: a first rail unit which is provided in a straight line shape to be close to an upper end of the housing; and a sliding unit having one end coupled to the first rail unit and a lower end connected to the transducer.

According to an exemplary embodiment of the present disclosure, the sliding unit may be extended and shortened in a vertical direction when the transducer moves in a horizontal direction through the first rail unit.

According to an exemplary embodiment of the present disclosure, the sliding unit may include: a first coupling unit which is coupled to the first rail unit to horizontally move along a straight line shape of the first rail unit; a guiding unit which is connected to a lower end of the first coupling unit and includes at least one sliding groove; a column unit which includes at least one protrusion member which is fitted into at least one sliding groove; and a second coupling unit which is connected to a lower end of the column unit and is connected to the transducer through a lower end.

According to an exemplary embodiment of the present disclosure, the movement module may further include: a driving motor which drives the sliding unit to be extended and shortened to ascend and descend the transducer in accordance with the curved shape, when the transducer moves in a horizontal direction.

According to an exemplary embodiment of the present disclosure, the movement module may further include: a second rail unit which is provided in a curved line shape corresponding to the curved shape to be close to the lower end of the housing, and the other end of the sliding unit may be coupled to the second rail unit.

According to an exemplary embodiment of the present disclosure, the transducer may ascend and descend along a curved line formed by the second rail unit by extending and shortening the sliding unit.

According to an exemplary embodiment of the present disclosure, the second coupling unit may be coupled to the second rail unit to move in a curved line in accordance with the curved shape of the second rail unit.

According to an exemplary embodiment of the present disclosure, the column unit and the second coupling unit are linked to the movement of the first coupling unit to move vertically and in a curved line, respectively.

According to an exemplary embodiment of the present disclosure, the sliding unit may be extended and shortened by vertically moving the column unit including at least one protrusion member which is fitted into at least one sliding groove.

As a technical means to achieve the above-described technical object, according to an aspect of the present disclosure, a driving method of an ultrasound probe including an ultrasound transducer which moves in a curved line may include a step of moving a transducer in a curved line in accordance with a curved shape of a contact surface which is in contact with a target area of a user; and a step of outputting an ultrasound signal from the transducer.

As a technical means to achieve the above-described technical object, according to an aspect of the present disclosure, an ultrasound probe including an ultrasound transducer which moves in a curved line may include a cartridge including a transducer which outputs an ultrasound signal and a movement module which moves the transducer in a curved line in accordance with a curved shape of a contact surface which is in contact with a target area of a user; and a handpiece from which the cartridge is detachable.

The above-described solving means are merely illustrative but should not be construed as limiting the present disclosure. In addition to the above-described embodiments, additional embodiments may be further provided in the drawings and the detailed description of the present disclosure.

### Advantageous Effects

According to the above-described solving means of the present disclosure, a transducer includes one surface which is in contact with a target area of the user and is formed in a curved shape and moves along the curved shape so that the ultrasound signal reaches the tissue layer at the same depth.

However, the effect which can be achieved by the present disclosure is not limited to the above-described effects, there may be another effect.

### DESCRIPTION OF DRAWINGS

FIG. 1 is a conceptual diagram of a driving system for an ultrasound probe including an ultrasound transducer which moves in a curved line according to an exemplary embodiment of the present disclosure.

FIG. 2 is a schematic block diagram of an ultrasound probe including an ultrasound transducer which moves in a curved line according to an exemplary embodiment of the present disclosure.

FIG. 3 is a structural diagram of an ultrasound probe including an ultrasound transducer which moves in a curved line according to an exemplary embodiment of the present disclosure.

FIG. 4 is a schematic block diagram of an ultrasound probe including an ultrasound transducer which moves in a curved line according to another exemplary embodiment of the present disclosure.

FIG. 5 is a structural diagram of an ultrasound probe including an ultrasound transducer which moves in a curved line according to another exemplary embodiment of the present disclosure.

FIG. 6 is a structural diagram illustrating an exemplary embodiment of ascending and descending an ultrasound transducer of an ultrasound probe including an ultrasound transducer which moves in a curved line according to another exemplary embodiment of the present disclosure.

FIG. 7 is an operation flowchart for a driving method of an ultrasound probe including an ultrasound transducer which moves in a curved line according to an exemplary embodiment of the present disclosure.

### BEST MODE

Hereinafter, the present disclosure will be described more fully with reference to the accompanying drawings, in which exemplary embodiments of the present disclosure are shown. However, the present disclosure can be realized in various different forms, and is not limited to the embodiments described herein. Accordingly, in order to clearly explain the present disclosure in the drawings, portions not related to the description are omitted. Like reference numerals designate like elements throughout the specification.

Throughout this specification and the claims that follow, when it is described that an element is “coupled” to another element, the element may be “directly coupled” to the other element or “electrically coupled” or “indirectly coupled” to the other element through a third element.

Through the specification of the present disclosure, when one member is located “on”, “above”, “on an upper portion”, “below”, “under”, and “on a lower portion” of the other member, the member may be adjacent to the other member or a third member may be disposed between the above two members.

In the specification of the present disclosure, unless explicitly described to the contrary, the word “comprise” and variations such as “comprises” or “comprising” will be understood to imply the inclusion of stated elements but not the exclusion of any other elements.

In the description of the exemplary embodiment of the present disclosure, terms regarding a direction or a position (an upper side, a top surface, or a lower side) are set with respect to a placement state of each component illustrated in the drawing.

The present disclosure relates to an ultrasound probe including an ultrasound transducer which moves in a curved line and a driving method thereof.

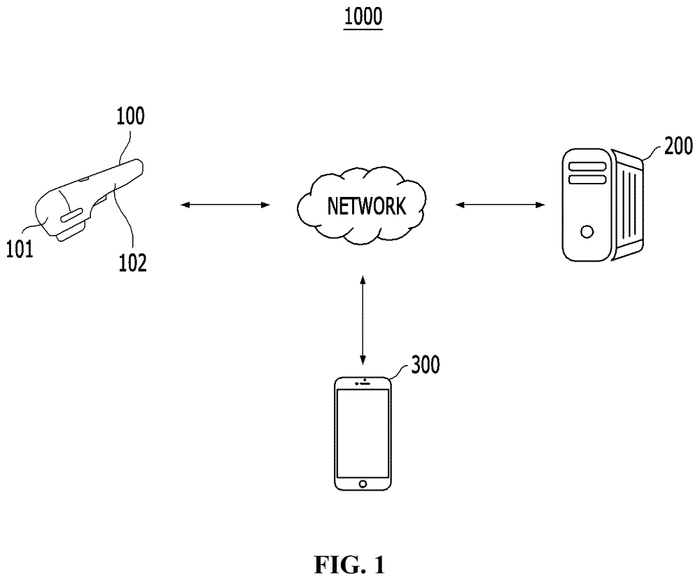

FIG. 1 is a conceptual diagram of a driving system for an ultrasound probe including an ultrasound transducer which moves in a curved line according to an exemplary embodiment of the present disclosure.

Referring to FIG. 1, a driving system 1000 for an ultrasound probe including an ultrasound transducer which moves in a curved line (hereinafter, referred to as a “driving system 1000”) may include an ultrasound probe 100 including an ultrasound transducer which moves in a curved line (hereinafter, referred to as a “ultrasound probe 100”) and a control device 200.

According to the exemplary embodiment of the present disclosure, the ultrasound probe 100 may irradiate an ultrasound signal to a focal point in a target area of a user 1. Generally, the ultrasound signal is transmitted into a tissue to be absorbed to be converted into heat energy. Specifically, the ultrasound having a sufficient energy may cause a rapid temperature rise in the tissue, which is referred to as a thermal effect of the ultrasound wave. According to this, the ultrasound probe 100 may irradiate the ultrasound signal to the target area of the user 1, to perform a procedure using a thermal effect of the ultrasound wave. Specifically, the high intensity focused ultrasound (HIFU) is an ultrasound wave in which the intensity is enhanced by focusing the ultrasound wave generated from the ultrasound transducer at a predetermined focal point.

Generally, the high intensity focused ultrasound does not directly act on the epidermal layer of the skin, but induces coagulation in the superficial musculo aponeurotic system (SMAS) which is a part of the muscle layer and delivers heat to the deeper region of the dermal layer, thereby not only reducing wrinkles, but also improving skin elasticity. According to the exemplary embodiment of the present disclosure, the ultrasound signal irradiated from the ultrasound probe 100 may be a high intensity focused ultrasound as described above.

Referring to FIG. 1, the ultrasound probe 100 may include a cartridge 101 and a handpiece 102. Here, the cartridge 101 is detachable from the handpiece 102 to be replaced to output an ultrasound signal according to a preference and a necessity of the user 1. A plurality of different cartridges having different ultrasound signal characteristics to be output may be provided. Further, the handpiece 102 may be held by the user 1.

According to the exemplary embodiment of the present disclosure, the control device 200 may control the ultrasound probe 100 and control the power on/off of the ultrasound probe 100 and the output of the ultrasound signal and the movement of the transducer 20.

The ultrasound probe 100 and the control device 200 may be linked via a network to share data therebetween. An example of the network may include a 3rd generation partnership project (3GPP) network, a long term evolution (LTE) network, a 5G network, a world interoperability for microwave access (WIMAX) network, wired/wireless Internet, a local area network (LAN), a wireless local area network (wireless LAN), a wide area network (WAN), a personal area network (PAN), a Bluetooth network, a Wi-Fi network, a near field communication (NFC) network, a satellite broadcasting network, an analog broadcasting network, and a digital multimedia broadcasting (DMB) network, but is not limited thereto.

Further, the control device 200 may be further linked to a terminal 300 which displays various monitoring information required for monitoring, such as a control state of the ultrasound probe 100, a skin condition of the user 1 which is expected to be improved by means of the use of the ultrasound probe 100, and recording data of the skin condition of the user 1. The terminal 300 may be all kinds of wireless communication devices, such as a smart phone, a smart pad, a tablet PC, a wearable device, a personal communication system (PCS), a global system for mobile communication (GSM), a personal digital cellular (PDC), a personal handyphone system (PHS), a personal digital assistant (PDA), an international mobile telecommunication (IMT)-2000, code division multiple access (CDMA)-2000, W-code division multiple access (W-CDMA), a wireless broadband internet (Wibro) terminal and fixed terminals such as a desktop computer and a smart TV.

Hereinafter, an internal structure of the ultrasound probe 100 according to the exemplary embodiment of the present disclosure will be described in detail.

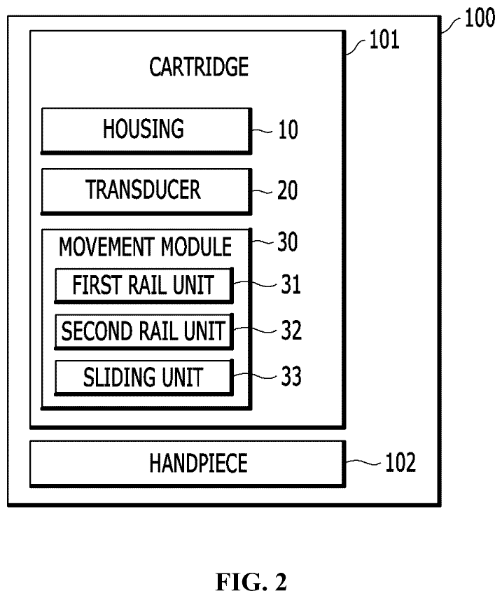

FIG. 2 is a schematic block diagram of an ultrasound probe including an ultrasound transducer which moves in a curved line according to an exemplary embodiment of the present disclosure.

Referring to FIG. 2, the ultrasound probe 100 may include a cartridge 101 and a handpiece 102 and the cartridge 101 may include a housing 10, a transducer 20, and a movement module 30. Further, the movement module 30 may include a first rail unit 31, a second rail unit 32, and a sliding unit 33.

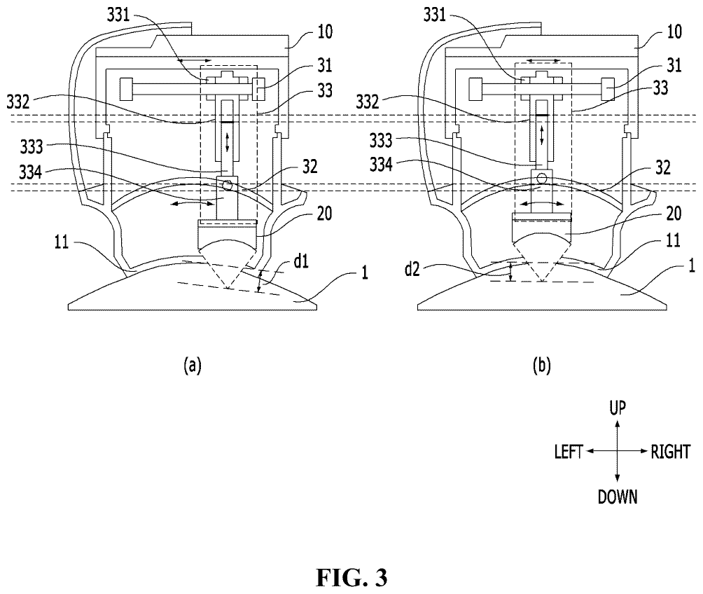

FIG. 3 is a diagram of a structure of an ultrasound probe including an ultrasound transducer which moves in a curved line according to an exemplary embodiment of the present disclosure.

Referring to FIG. 3, the housing 10 forms an outer appearance of the cartridge 101 and may include a contact surface 11 which is in contact with a target area of the user 1. At this time, the contact surface 11 may be provided in a curved shape so as to allow stable close contact with the target area of the user 1.

Further, referring to FIG. 3, the transducer 20 and the movement module 30 may be provided in the housing 10. The transducer 20 may output an ultrasound signal. Further, the movement module 30 may move the transducer 20 in a curved line along a curved shape of the contact surface 11.

With regard to this, referring to FIGS. 2 and 3, the movement module 30 may include a first rail unit 31, a second rail unit 32, and a sliding unit 33.

Referring to FIG. 3, the first rail unit 31 may be fixedly disposed at an upper end side of the housing 10 and may be provided in a straight line shape. At this time, the upper end side may refer to a side in the housing 10 which is relatively distant from the contact surface 11.

Further, referring to FIG. 3, the second rail unit 32 may be fixedly disposed at a lower end side of the housing 10 and may be provided in a curved line shape corresponding to the curved shape of the contact surface 11. That is, a curvature of the curved shape of the contact surface 11 and a curvature of the curved shape of the second rail unit 32 may be equal to each other. At this time, the lower end side may refer to a side in the housing 10 which is relatively close to the contact surface 11.

Further, referring to FIG. 3, one end of the sliding unit 33 may be coupled to the first rail unit 31 and the other end may be coupled to the second rail unit 32. At this time, one end may be close to the upper end of the sliding unit 33 and the other end may be close to the lower end of the sliding unit 33.

Specifically, the sliding unit 33 may include a first coupling unit 331, a guiding unit 332, a column unit 333, and a second coupling unit 334.

Referring to FIG. 3, the first coupling unit 331 may be coupled to the first rail unit 31 to horizontally move along a straight line shape of the first rail unit 31. With regard to this, the ultrasound probe 100 may further include a driver (not illustrated) which drives the horizontal movement of the first coupling unit 331 based on the user's setting.

Specifically, the driver (not illustrated) may be provided in the cartridge 101 or the handpiece 102, and for example, may be a linear motor, but is not limited thereto. Further, the user setting may be transmitted through the control device 200 and may be set based on user input through at least one button (not illustrated) provided in the handpiece 102.

Referring to FIG. 3, the guiding unit 332 may include at least one sliding groove and may be connected to the lower end of the first coupling unit 331. At this time, at least one sliding groove may be provided as a hollow in which an inside of the guiding unit 332 is empty, as illustrated in FIG. 3. However, the sliding groove is not limited thereto and may be provided in various types of grooves which are fastened with a protrusion member to be described below.

Further, referring to FIG. 3, the column unit 333 may include at least one protrusion member which is fitted into at least one sliding groove. At this time, when the sliding groove is provided as a hollow as illustrated in FIG. 3, the column unit 333 does not additionally include a separate protrusion member, but the column unit 333 itself may serve as a protrusion member.

However, the column unit is not limited thereto and may be provided in various forms to be fastened with the sliding groove. For example, when the sliding groove is a linear groove which extends in a vertical direction, the protrusion member may be provided in a ring shape which may be caught by the sliding groove or in a bar shape which is properly fitted into the sliding groove.

With regard to this, the sliding unit 33 may be extended and shortened in the vertical direction and by means of vertical movement of the column unit 333 including at least one protruding member which is fitted into at least one sliding groove.

Further, referring to FIG. 3, the second coupling unit 334 may be coupled to the second rail unit 32 to move in a curved line along the curved shape of the second rail unit 32 and be connected to a lower end of the column unit 333. Specifically, the second coupling unit 334 may include a roller member so as to easily move along the second rail unit 32. Further, the second coupling unit 334 is linked with the first coupling unit 331 which is driven by a driver (not illustrated) to move along the second rail unit 32.

Specifically, (a) of FIG. 3 illustrates a state in which the first coupling unit 331 is located in one end (a right end in (a) of FIG. 3) of the first rail unit 31 so that the transducer 20 relatively descends and (b) of FIG. 3 illustrates a state in which the first coupling unit 331 is located in a central portion of the first rail unit 31, that is, the second coupling unit 334 is located in a bent point of the second rail unit 32 so that the transducer 20 relatively ascends.

Referring to FIG. 3, if the first coupling unit 331 moves from the right end (in (a) of FIG. 3) to the central portion ((b) of FIG. 3) by the driver (not illustrated) (moves from (a) of FIG. 3 to (b) of FIG. 3, that is, to the left side), the guiding unit 332 connected to the first coupling unit 331 may also move to the left side together with the first coupling unit 331. Accordingly, the second coupling unit 334 including the roller member may move to the bent point of the second rail unit 32 along the second rail unit 32. At this time, as the second coupling unit 334 moves to the bent point of the second rail unit 32, not only the position may move to the central portion, but also the height may also be increased so as to correspond to the curvature of the curved shape of the second rail unit 32.

Here, the column unit 333 may ascend along the sliding groove of the guiding unit 332 so as to correspond to the curvature of the curved shape of the second rail unit 32 to move the position of the second coupling unit 334.

That is, when the first coupling unit 331 horizontally moves by the driver (not illustrated) in a horizontal direction, the guiding unit 332 connected to the first coupling unit 331 may also horizontally move together with the first coupling unit 331. Accordingly, the column unit 333 may vertically move as much as a height corresponding to a movement distance of the first coupling unit 331 and the curvature of the second rail unit 32 and the second coupling unit 334 may also move along the curved line, along the second rail unit 32 so as to correspond to the movement distance of the first coupling unit 331.

In other words, the first coupling unit 331, the guiding unit 332, the column unit 333, and the second coupling unit 334 are linked to each other so that even though the driver (not illustrated) drives only the movement of the first coupling unit 331, the guiding unit 332, the column unit 333, and the second coupling unit 334 may also horizontally move, vertically move, and move in the curved line, respectively, in accordance with the movement of the first coupling unit 331.

However, the driver is not limited thereto and the driver (not illustrated) may further drive not only the first coupling unit 331, but also at least one of the guiding unit 332, the column unit 333, and the second coupling unit 334 and drive only the second coupling unit 334, instead of the first coupling unit 331.

In other words, as the transducer 20 moves in a horizontal direction, the sliding unit 33 may be extended and shortened in the vertical direction by means of the first rail unit 31 and the second rail unit 32. Further, the transducer 20 may ascend and descend along the curved line formed by the second rail unit 32 by the extension and the shortening of the sliding unit 33.

According to this, the ultrasound probe 100 includes a transducer which includes one surface which contacts with the target area of the user and is formed in a curved shape and moves along the curved line in accordance with the curved shape. Therefore, when an ultrasound signal is focused in the target area of the user 1 which is pressed in a curved shape along the contact surface 11 provided in a curved shape to closely contact with the contact surface 11, the height of the transducer 20 is adjusted along the second rail unit 32 having a curvature corresponding to the curved shape of the contact surface 11, thereby allowing the ultrasound signal to reach the tissue layer at the same depth.

Referring to FIG. 3, it is confirmed that a distance d1 from the skin surface of the target area of the user 1 to a focal point at which the ultrasound signal is focused when the transducer 20 is located at the right end in the housing 10 in (a) of FIG. 3 is equal to a distance d2 from the skin surface of the target area of the user 1 to the focal point at which the ultrasound signal is focused when the transducer 20 is located in the central portion in the housing 10 in (b) of FIG. 3.

That is, the ultrasound probe 100 may focus the ultrasound signal at the focal point of the tissue layer of the target area of the user 1 while moving the position of the transducer 20 and in this case, even though the position of the transducer 20 varies, focus the ultrasound signal at the focal point with a constant focusing depth.

Hereinafter, an internal structure of the ultrasound probe 100 according to another exemplary embodiment of the present disclosure will be described in detail.

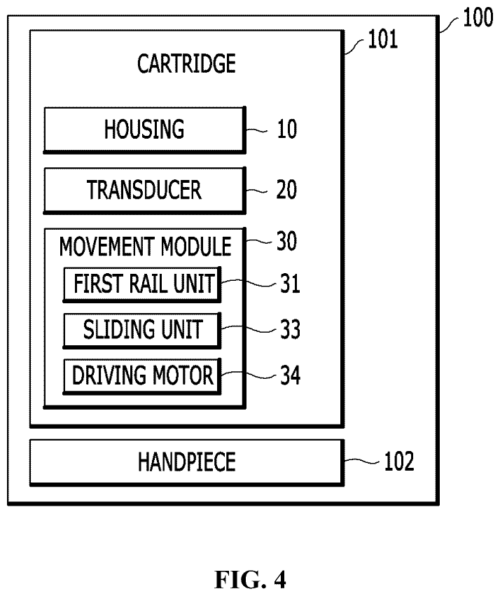

FIG. 4 is a schematic block diagram of an ultrasound probe including an ultrasound transducer which moves in a curved line according to another exemplary embodiment of the present disclosure.

Referring to FIG. 4, the ultrasound probe 100 may include a cartridge 101 and a handpiece 102 and the cartridge 101 may include a housing 10, a transducer 20, and a movement module 30. Further, the movement module 30 may include a first rail unit 31, a sliding unit 33, and a driving motor 34.

Hereinafter, the cartridge 101, the handpiece 102, the housing 10, the transducer 20, and the first rail unit 31 of the movement module 30 are the same configurations as the exemplary embodiment of the present disclosure which have been described with reference to FIGS. 2 and 3, so that a redundant description will be omitted.

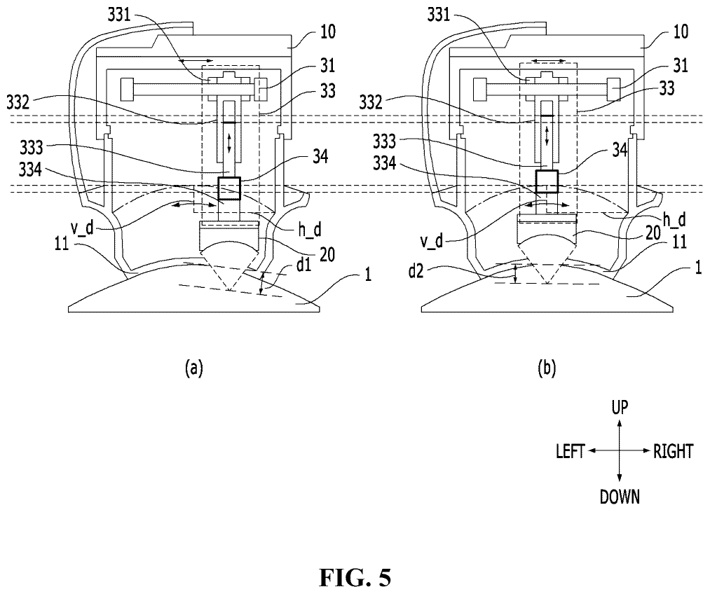

FIG. 5 is a diagram of a structure of an ultrasound probe including an ultrasound transducer which moves in a curved line according to another exemplary embodiment of the present disclosure.

Referring to FIGS. 4 and 5, the movement module 30 may include the driving motor 34. The driving motor 34 may drive the sliding unit 330 to be extended and shortened so as to ascend or descend the transducer 20 along the curved shape of the housing 10 when the transducer 20 moves in a horizontal direction. For example, the driving motor 34 is provided at one side of the second coupling unit 334 to linearly move the transducer 20 connected to the second coupling unit 334 in a vertical direction (up-down direction). Accordingly, the driving motor 34 may be a high speed linear motor, for example, but is not limited thereto.

Specifically, the driving motor 34 may adjust the extension and the shortening of the sliding unit 33 based on a control signal according to the user setting which is transmitted through the control device 200. At this time, the control signal may include a signal about a vertical movement distance v_d at which the sliding unit 33 is extended and shortened, in accordance with the horizontal movement distance h_d at which the first coupling unit 331 is coupled to the first rail unit 31 to move horizontally and the horizontal movement direction.

Referring to FIG. 5, even though the second rail unit 32 according to the exemplary embodiment is not present, when the first coupling unit 331 moves, the driving motor 34 is linked to the horizontal movement distance h_d and the horizontal movement distance of the first coupling unit 331 to drive the extension and the shortening of the sliding unit 33 as much as a predetermined vertical movement distance v_d, thereby ascending and descending the transducer 20 along the curved shape of the contact surface 11.

As this time, unlike the above-described exemplary embodiment, the second coupling unit 334 is not coupled to the second rail unit 32, but only the transducer 20 is connected to the lower end thereof to support the transducer 20. Further, the guiding unit 332 and the column unit 333 may be provided to have the same as or similar to the above-described exemplary embodiment, but specifically, the vertical movement of the column unit 333 may be performed by the driving motor 34.

According to this, the ultrasound probe 100 includes a transducer which includes one surface which contacts with the target area of the user and is formed in a curved shape and moves along the curved line in accordance with the curved shape. Therefore, when an ultrasound signal is focused in the target area of the user 1 which is pressed in a curved shape along the contact surface 11 provided in a curved shape to closely contact with the contact surface 11, a vertical movement distance v_d of the transducer 20 in accordance with the horizontal movement distance h_d and the horizontal movement direction of the transducer 20 is adjusted using the driving motor 34 so as to correspond to the curved shape of the contact surface 11, thereby allowing the ultrasound signal to reach the tissue layer at the same depth.

Referring to FIG. 5, it is confirmed that a distance d1 from the skin surface of the target area of the user 1 to a focal point at which the ultrasound signal is focused when the transducer 20 is located at the right end in the housing 10 in (a) of FIG. 5 is equal to a distance d2 from the skin surface of the target area of the user 1 to the focal point at which the ultrasound signal is focused when the transducer 20 is located in the central portion in the housing 10 in (b) of FIG. 5.

That is, the ultrasound probe 100 may focus the ultrasound signal at the focal point of the tissue layer of the target area of the user 1 while moving the position of the transducer 20 and in this case, even though the position of the transducer 20 varies, focus the ultrasound signal at the focal point with a constant focusing depth.

However, the present disclosure is not limited thereto and as illustrated in FIG. 5, when the second rail unit 32 is not present, the ultrasound probe 100 may further increase the ascending and descending range of the transducer 20 so as to form a focal point in various depths in addition to a depth according to the curved-movement along the curved shape corresponding to the contact surface 11 by extending and shortening the sliding unit 33 in accordance with the control signal.

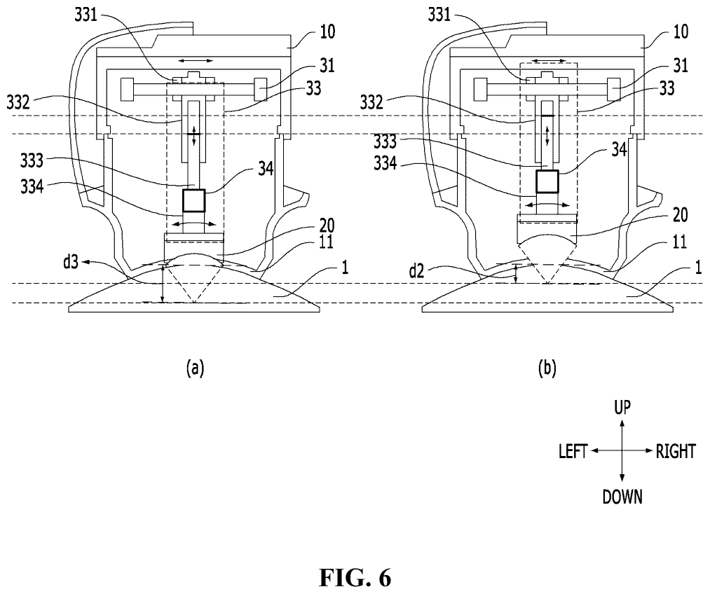

FIG. 6 is a structural diagram illustrating an exemplary embodiment of ascending and descending an ultrasound transducer of an ultrasound probe including an ultrasound transducer which moves in a curved line according to another exemplary embodiment of the present disclosure.

Referring to FIG. 6, the driving motor 34 adjusts a vertical movement of the transducer 20 in accordance with the control signal according to the user setting transmitted through the control device 200 to adjust a distance from the skin surface of the target area of the user 1 to a focal point at which the ultrasound signal is focused.

Accordingly, the driving motor 34 may adjust a vertical position of the transducer 20 so as to vary a depth of the focal point to a distance d3 from the skin surface of the target area of the user 1 as illustrated in (a) of FIG. 6 to the focal point at which the ultrasound signal is focused which is longer than a distance d2 from the skin surface of the target area of the user 1 illustrated in FIGS. 3 and 5 as illustrated in (b) of FIG. 6 to the focal point at which the ultrasound signal is focused.

Accordingly, the ultrasound probe 100 may also adjust the depth of the ultrasound focal point according to the user setting transmitted through the control device 200, without being mechanically restricted by the curved shape of the contact portion of the target area.

Hereinafter, an operation flow of the present disclosure will be described in brief based on the above detailed description.

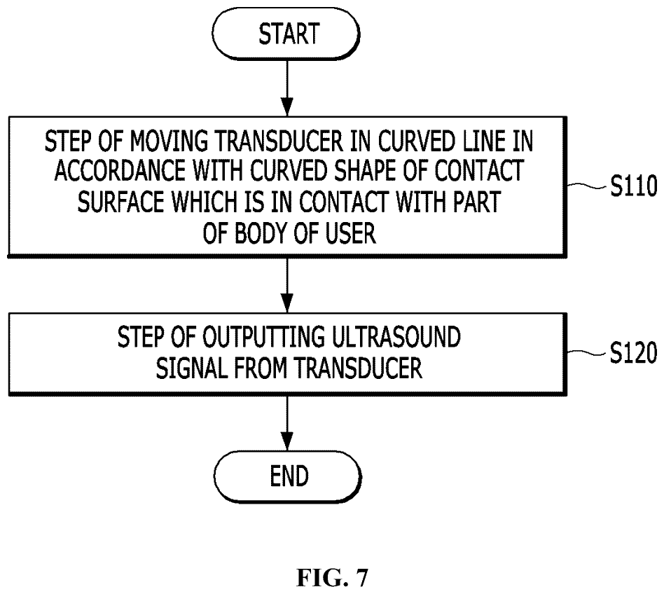

FIG. 7 is an operation flowchart for a driving method of an ultrasound probe including an ultrasound transducer which moves in a curved line according to an exemplary embodiment of the present disclosure.

The driving method of an ultrasound probe including an ultrasound transducer which moves in a curved line illustrated in FIG. 7 may be performed by the above-described ultrasound probe 100 including an ultrasound transducer which moves in a curved line. Accordingly, even though it is not described below, the contents described for the ultrasound probe 100 including an ultrasound transducer which moves in a curved line may also be applied to the description for the driving method of an ultrasound probe including an ultrasound transducer which moves in a curved line in the same way.

Referring to FIG. 7, in step S110, the transducer 20 may be included in the housing 10 and move along a curved line in accordance with the curved shape of the contact surface 11 which is in contact with the target area of the user 1. At this time, the transducer 20 may be provided in the housing 10.

In the meantime, the movement module 30 may be provided in the housing 10 and the movement module 30 may move the transducer 20 in a curved line along the curved shape of the contact surface 11.

With regard to this, the movement module 30 may include a first rail unit 31, a second rail unit 32, and a sliding unit 33. The first rail unit 31 may be provided in a straight line shape to be close to the upper end of the housing 10. The second rail unit 32 may be provided close to the lower end of the housing 10 in a curved line shape corresponding to the curved shape of the contact surface 11. One end of the sliding unit 33 may be coupled to the first rail unit 31 and the other end may be coupled to the second rail unit 32.

Further, as the transducer 20 moves in a horizontal direction, the sliding unit 33 may be extended and shortened in the vertical direction by means of the first rail unit 31 and the second rail unit 32.

Specifically, the sliding unit 33 may include a first coupling unit 331, a guiding unit 332, a column unit 333, and a second coupling unit 334.

The first coupling unit 331 may be coupled to the first rail unit 31 to horizontally move along a straight line shape of the first rail unit 31. The guiding unit 332 may be connected to the lower end of the first coupling unit 331 and include at least one sliding groove. The column unit 333 may include at least one protrusion member which is fitted into at least one sliding groove. Further, the second coupling unit 334 may be connected to the lower end of the column unit 333 and may be coupled to the second rail unit 32 to move in a curved line along the curved shape of the second rail unit 32.

At this time, the first coupling unit 331 may move horizontally along the first rail unit 31 by the driver (not illustrated) which is driven based on the user setting and the column unit 333 and the second coupling unit 334 may be linked to the movement of the first coupling unit 331 to move vertically and in a curved line.

In other words, the sliding unit 33 may be extended and shortened by the vertical movement of the column unit 333 including at least one protrusion member which is fitted into at least one sliding groove. Further, the transducer 20 may ascend and descend along the curved line formed by the second rail unit 32 by the extension and the shortening of the sliding unit 33.

Next, in step S120, the ultrasound signal may be output from the transducer 20.

In the above description, steps S110 and S120 may be further divided into additional steps or combined as smaller steps depending on an implementation example of the present disclosure. Further, some steps may be omitted if necessary and the order of steps may be changed.

The driving method of an ultrasound probe including an ultrasound transducer which moves in a curved line according to the exemplary embodiment of the present disclosure may be implemented as a program command which may be executed by various computer means to be recorded in a computer readable medium. The computer readable medium may include solely a program command, a data file, and a data structure or a combination thereof. The program commands recorded in the medium may be specifically designed and constructed for the present disclosure or known to those skilled in the art of a computer software to be used. Examples of the computer readable recording medium include magnetic media such as a hard disk, a floppy disk, or a magnetic tape, optical media such as a CD-ROM or a DVD, magneto-optical media such as a floptical disk, and a hardware device which is specifically configured to store and execute the program command such as a ROM, a RAM, and a flash memory. Examples of the program command include not only a machine language code which is created by a compiler but also a high level language code which may be executed by a computer using an interpreter. The hardware device may operate as one or more software modules in order to perform the operation of the present disclosure and vice versa.

Further, the above-described driving method of an ultrasound probe including an ultrasound transducer which moves in a curved line may also be implemented as a computer program or an application executed by a computer which is stored in a recording medium.

The above description of the present disclosure is illustrative only and it is understood by those skilled in the art that the present disclosure may be easily modified to another specific type without changing the technical spirit of an essential feature of the present disclosure. Thus, it is to be appreciated that embodiments described above are intended to be illustrative in every sense, and not restrictive. For example, each component which is described as a singular form may be divided to be implemented and similarly, components which are described as a divided form may be combined to be implemented.

The scope of the present disclosure is represented by the claims to be described below rather than the detailed description, and it is to be interpreted that the meaning and scope of the claims and all the changes or modified forms derived from the equivalents thereof come within the scope of the present disclosure.

### Explanation of Reference Numerals and Symbols

1000: Driving system for ultrasound probe including ultrasound transducer which moves in curved line
100: Ultrasound probe including ultrasound transducer which moves in curved line
101: Cartridge
102: Handpiece
10: Housing
11: Contact surface
20: Transducer
30: Movement module
31: First rail unit
32: Second rail unit
33: Sliding unit
331: First coupling unit
332: Guiding unit
333: Column unit
334: Second coupling unit
34: Driving motor
200: Control device
1: User

1. An ultrasound probe including an ultrasound transducer which moves in a curved line, comprising:
a housing including a contact surface which is provided in a curved shape and is in contact with a target area of a user;
a transducer which is provided in the housing and outputs an ultrasound signal; and
a movement module which moves the transducer in the curved line in accordance with the curved shape.

2. The ultrasound probe of claim 1, wherein the movement module includes:
a first rail unit which is provided in a straight line shape to be close to an upper end of the housing; and
a sliding unit having one end coupled to the first rail unit and a lower end connected to the transducer.

3. The ultrasound probe of claim 2, wherein the sliding unit is extended and shortened in a vertical direction when the transducer moves in a horizontal direction through the first rail unit.

4. The ultrasound probe of claim 3, wherein the sliding unit includes:
a first coupling unit which is coupled to the first rail unit to horizontally move along the straight line shape of the first rail unit;
a guiding unit which is connected to a lower end of the first coupling unit and includes at least one sliding groove;
a column unit which includes at least one protrusion member which is fitted into the at least one sliding groove; and
a second coupling unit which is connected to a lower end of the column unit and is connected to the transducer through a lower end.

5. The ultrasound probe of claim 4, wherein the movement module further includes:
a driving motor which drives the sliding unit to be extended and shortened to ascend and descend the transducer in accordance with the curved shape, when the transducer moves in the horizontal direction.

6. The ultrasound probe of claim 4, wherein the movement module further includes:
a second rail unit which is provided in a curved line shape corresponding to the curved shape to be close to a lower end of the housing,
wherein the other end of the sliding unit is coupled to the second rail unit.

7. The ultrasound probe of claim 6, wherein the transducer ascends and descends along the curved line formed by the second rail unit by extending and shortening the sliding unit.

8. The ultrasound probe of claim 6, wherein the second coupling unit is coupled to the second rail unit to move in the curved line in accordance with the curved shape of the second rail unit.

9. The ultrasound probe of claim 8, wherein the column unit and the second coupling unit are linked to movement of the first coupling unit to move vertically and in the curved line, respectively.

10. The ultrasound probe of claim 4, wherein the sliding unit is extended and shortened by vertically moving the column unit including the at least one protrusion member which is fitted into the at least one sliding groove.

11. A driving method of an ultrasound probe including an ultrasound transducer which moves in a curved line, comprising:
a step of moving a transducer in the curved line in accordance with a curved shape of a contact surface which is in contact with a target area of a user; and
a step of outputting an ultrasound signal from the transducer.

12. An ultrasound probe including an ultrasound transducer which moves in a curved line, comprising:
a cartridge including a transducer which outputs an ultrasound signal and a movement module which moves the transducer in the curved line in accordance with a curved shape of a contact surface which is in contact with a target area of a user; and
a handpiece from which the cartridge is detachable.

13. A computer readable recording medium recording a program which allows a computer to execute a method according to claim 11.
